(19) United States
(12) Patent Application Publication
(10) Pub. No.: US 2026/0306093 A1
(43) Pub. Date: Oct. 8, 2026

(54) SUTURE ANCHOR DEVICES, SYSTEMS, AND METHODS

(71) Applicant: NovantX, LLC, Tampa, FL (US)

(72) Inventors: Sergio Gutiérrez, Tampa, FL (US); Jonathan Chad Levy, Ft. Lauderdale, FL (US)

(73) Assignee: NovantX, LLC, Tampa, FL (US)

(21) Appl. No.: 19/642,498

(22) Filed: Apr. 8, 2026

Related U.S. Application Data
(60) Provisional application No. 63/785,140, filed on Apr. 8, 2025.

(51) Int. Cl. A61B 17/04

(52) U.S. Cl. CPC A61B 17/0401; A61B 2017/0409; A61B 2017/044; A61B 2017/0453

(57) ABSTRACT

An assembly for attaching tissue to bone may include a driver handle, a driver shaft, a driver engagement member, and a driver tip. The driver handle may include a longitudinal handle passageway and may couple with the driver shaft which has a longitudinal shaft passageway. The driver engagement member may couple with the driver shaft and include a driver torque connection feature and a driver channel. The driver tip may couple with the driver engagement member and protrude from a distal end of an anchor coupled with the driver engagement member. A flexible element may couple with the anchor and pass through the anchor and into the longitudinal shaft passageway along an interior space formed between an inner wall of the anchor and the driver channel, pass through the longitudinal shaft passageway, pass through the longitudinal handle passageway, and exit out of a proximal end of the driver handle.

### CROSS-REFERENCE TO RELATED APPLICATIONS

This application claims the benefit of U.S. Provisional Patent Application Ser. No. 63/785,140 filed on Apr. 8, 2025, entitled “SELF STARTING AND TAPPING SUTURE ANCHOR SYSTEM AND METHODS,” which is incorporated by reference as though set forth herein in its entirety.

### TECHNICAL FIELD

The present disclosure relates to suture anchor devices, systems, and methods. More specifically, the present disclosure relates to suture anchor devices, systems, and methods for attaching soft tissue to bone during a surgical procedure including, but not limited to: tendon repair, ligament repair, labral repair, other musculoskeletal repairs/reconstructions, etc. The implantable fixation devices and instruments described herein can provide immediate mechanical fixation, promote long-term biological integration with bone and other tissues, and enable efficient suture management during the surgical procedure.

### BACKGROUND

Surgical procedures, especially those involving the repair or reattachment of soft tissue to bone (e.g., in rotator cuff repair, labral repair, tendon reconstructions, etc.), can benefit from robust fixation solutions. For example, a surgeon may place a suture anchor into a patient's bone to secure one or more sutures to hold or approximate a torn soft tissue to the bone. However, conventional suture anchors and anchor drivers present may challenges such as poor bone purchase, complicated suture management, insufficient integration with surrounding bone, etc.

Soft tissue injuries that require reattachment of the soft tissue to bone are common in the orthopedic space. Traditional treatments have evolved from open surgical techniques to arthroscopic approaches that utilize one or more suture anchors to serve as attachment points for sutures that secure soft tissue to bone. Despite advancements from early metallic designs to current sophisticated systems, several challenges persist in clinical practice.

Current suture anchor technologies face three primary limitations. First, fixation of the suture anchor within poor-quality bone remains problematic, particularly in osteoporotic patients where anchor loosening or anchor pull-out is a concern. Second, managing multiple sutures during the arthroscopic procedure is technically demanding, often resulting in tangling, misidentification, and/or suboptimal suture tensioning. Third, conventional anchor designs frequently lack features that promote long-term biological integration with surrounding bone.

Additional challenges include the technical complexity of anchor insertion, with many designs requiring pre-drilled pilot holes that can potentially weaken the bone-anchor interface, and also require a matched trajectory during anchor placement that may not provide optimal fixation. Self-punching anchor designs exist, but material and/or design limitations for anchors often result in poor advancing dynamics of the anchor into bone and/or require a metallic punching element to be left in the bone after the procedure. Furthermore, other know fixation mechanisms (e.g., threaded, press-fit, toggle-type anchors, etc.) each present their own inherent limitations, with none of these designs offering an ideal combination of ease of insertion, immediate stability, and long-term bone integration for the anchor.

Accordingly, improved suture anchors and anchor drivers that result in secure fixation for the anchor across varying bone densities, facilitate simpler suture management processes, enable easy anchor insertion without the need to pre-drill or pre-punch a hole in the bone, promote long-term osseointegration (particularly in challenging anatomical or bone-quality conditions), etc., would be desirable.

### SUMMARY

The suture anchor devices, systems, and methods of the present disclosure have been developed in response to the present state of the art, and in particular, in response to the problems and needs in the art that have not yet been fully solved by currently available suture anchor devices, systems, and methods. In some embodiments, the suture anchor devices, systems, and methods of the present disclosure may provide improved devices, systems, and methods for attaching soft tissue to bone. It will be understood that the below summary is illustrative only and should not be regarded as limiting.

In some embodiments, an assembly for attaching tissue to bone may include an anchor driver, an anchor, and at least one flexible element. The anchor driver may include a driver shaft having: a proximal end, a distal end, a longitudinal axis, and a longitudinal shaft passageway formed through the driver shaft between the proximal end and the distal end of the driver shaft. The anchor driver may also include a driver engagement member proximate the distal end of the driver shaft that may include at least one driver torque connection feature and at least one driver channel. The anchor driver may also include a driver tip at a distal end of the driver engagement member. The anchor may be configured to removably couple with the driver engagement member and may include: a proximal end, a distal end, a longitudinal axis, a longitudinal anchor passageway formed through the anchor between the proximal end and the distal end of the anchor that defines an inner wall within the anchor, and at least one anchor torque connection feature configured to engage with the at least one driver torque connection feature and receive a torque force transmitted therefrom. The at least one flexible element may be configured to couple with the anchor such that, when the anchor is coupled to the driver engagement member of the anchor driver, and the flexible element is coupled to the anchor: the driver tip may be configured to protrude from the distal end of the anchor; the flexible element may be configured to pass through the anchor and into the longitudinal shaft passageway along an interior space that may be formed between the inner wall of the anchor and the driver channel; and the flexible element may be configured to pass through the longitudinal shaft passageway and exit the proximal end of the driver shaft.

The assembly according to any preceding paragraph, wherein: the driver torque connection feature may include a plurality of driver torque connection features; the anchor torque connection feature may include a plurality of anchor torque connection features configured to engage with the plurality of driver torque connection features and receive the torque force transmitted therefrom; the driver channel may include a plurality of driver channels; the flexible element may include a plurality of flexible elements configured to couple with the anchor; the plurality of flexible elements may be configured to pass through the anchor and into the longitudinal shaft passageway along a plurality of interior spaces that may be formed between the inner wall of the anchor and the plurality of driver channels; and the plurality of flexible elements may be configured to pass through the longitudinal shaft passageway and exit the proximal end of the driver shaft.

The assembly according to any preceding paragraph, wherein the driver tip may include a driver tip shank and at least one driver tip channel formed in the driver tip shank.

The assembly according to any preceding paragraph, wherein the driver tip shank may include at least one driver tip thread disposed about the driver tip shank.

The assembly according to any preceding paragraph, wherein at least a portion of the driver tip shank may include a constant width.

The assembly according to any preceding paragraph, wherein the driver tip shank may include a tapered shape.

The assembly according to any preceding paragraph, wherein the anchor may include at least one bridge that may be configured to couple the flexible element to the anchor.

In some embodiments, an assembly for attaching tissue to bone may include a driver handle, a driver shaft, a driver engagement member, and a driver tip. The driver handle may include: a proximal end, a distal end, a longitudinal axis, and a longitudinal handle passageway formed through the driver handle between the proximal end and the distal end of the driver handle. The driver shaft may include a proximal end coupled to or integrated with the distal end of the driver handle, a distal end, a longitudinal axis, and a longitudinal shaft passageway formed through the driver shaft between the proximal end and the distal end of the driver shaft. The driver engagement member may be proximate the distal end of the driver shaft and may include a driver torque connection feature and a driver channel. The driver tip may be at a distal end of the driver engagement member and may be configured to protrude from a distal end of an anchor that may be coupled with the driver engagement member. A flexible element may be coupled to the anchor and configured to pass through the anchor and into the longitudinal shaft passageway along an interior space that may be formed between an inner wall of the anchor and the driver channel. The flexible element may also be configured to pass through the longitudinal shaft passageway and exit the proximal end of the driver shaft. The flexible element may be further configured to pass through the longitudinal handle passageway and exit the proximal end of the driver handle.

The assembly according to any preceding paragraph, wherein: the driver torque connection feature may include a plurality of driver torque connection features configured to engage a plurality of anchor torque connection features formed within the anchor and transmit a torque force to the anchor; the driver channel may include a plurality of driver channels; the flexible element may include a plurality of flexible elements coupled to the anchor; the plurality of flexible elements may be configured to pass through the anchor and into the longitudinal shaft passageway along a plurality of interior spaces that may be formed between the inner wall of the anchor and the plurality of driver channels; the plurality of flexible elements may be configured to pass through the longitudinal shaft passageway and exit the proximal end of the driver shaft; and the plurality of flexible elements may be configured to pass through the longitudinal handle passageway and exit the proximal end of the driver handle.

The assembly according to any preceding paragraph, wherein the proximal end of the driver handle may include a slot formed therein that may be configured to receive the flexible element therein and guide the flexible element along a transverse orientation relative to the proximal end of the driver handle.

The assembly according to any preceding paragraph, wherein the slot may include a plurality of slots formed in the proximal end of the driver handle that may be configured to receive a plurality of flexible elements therein to guide the plurality of flexible elements along a plurality of transverse orientations relative to the proximal end of the driver handle.

The assembly according to any preceding paragraph, wherein the distal end of the driver handle may include an O-ring configured to secure the flexible element to the driver handle.

The assembly according to any preceding paragraph, wherein the driver handle may include a flexible element retaining mechanism disposed on a first side of the driver handle.

The assembly according to any preceding paragraph, wherein the flexible element retaining mechanism may include a first plurality of flexible element retaining mechanisms disposed on the first side of the driver handle, and the driver handle may include a second plurality of flexible element retaining mechanisms disposed on a second side of the driver handle.

The assembly according to any preceding paragraph, wherein the flexible element retaining mechanism may include a locking mechanism that may be configured to grasp and lock the flexible element to the flexible element retaining mechanism.

In some embodiments, an assembly for attaching tissue to bone may include a flexible element and an anchor. The flexible element may include a first portion, a second portion, and a third portion. The anchor may include: an anchor body, a proximal end, a distal end, a longitudinal axis, a longitudinal anchor passageway formed through the anchor between the proximal end and the distal end of the anchor that defines an inner wall within the anchor, an anchor torque connection feature formed within the anchor, a proximal eyelet formed through the anchor, a distal eyelet formed through the anchor distal to the proximal eyelet, and a bridge positioned intermediate the proximal eyelet and the distal eyelet. The flexible element may be configured to pass through the proximal eyelet and the distal eyelet to couple the flexible element to the bridge at the third portion of the flexible element that may be engaged with the bridge. The first and second portions of the flexible element may be configured to pass through the anchor along an interior space that may be formed between the inner wall of the anchor and a driver channel of a driver engagement member that may be placed within the longitudinal anchor passageway, such that the first and second portions of the flexible element may exit from the proximal end of the anchor. The first and second portions of the flexible element may be configured to enter into and pass through a longitudinal shaft passageway formed through a driver shaft that may be coupled to or integrated with the driver engagement member, such that the first and second portions of the flexible element may exit from the proximal end of the driver shaft.

The assembly according to any preceding paragraph, wherein the anchor may include an anchor thread disposed about the anchor body.

The assembly according to any preceding paragraph, wherein at least one of the anchor body and the anchor thread may include a tapered shape.

The assembly according to any preceding paragraph, wherein the bridge may include a bridge recess formed on an outer surface of the anchor body, and a bridge protrusion formed on the inner wall within the anchor that may transversely protrude into the longitudinal anchor passageway formed through the anchor.

The assembly according to any preceding paragraph, wherein the anchor body may include at least one fenestration formed therethrough.

In some embodiments, a method for attaching soft tissue to bone may include: inserting at least one flexible element through a longitudinal anchor passageway that may be formed through an anchor; engaging the anchor with an anchor driver that comprises at least one flexible element retaining mechanism disposed thereon; securing the at least one flexible element to the at least one flexible element retaining mechanism; positioning a driver tip that may be disposed on a distal end of the anchor driver against a bone at a desired attachment site on the bone; applying force to the anchor driver to insert the anchor into the bone; and disengaging the anchor driver from the anchor, thereby leaving the anchor and the at least one flexible element in place within the bone for attaching the soft tissue thereto.

The method according to any preceding paragraph further including: guiding the at least one flexible element through a longitudinal shaft passageway that may be formed through a driver shaft of the anchor driver.

The method according to any preceding paragraph further including: penetrating cortical bone with a trocar tip of the driver tip that may be configured to extend beyond a distal end of the anchor when the anchor is coupled to the anchor driver.

The method according to any preceding paragraph further including: managing a plurality of flexible elements by securing the plurality of flexible elements to a plurality of flexible element retaining mechanisms.

In some embodiments, a system for attaching tissue to bone may include an implantable anchor and an anchor driver. The implantable anchor may include one or more fenestrations formed through the implantable anchor configured to promote biological integration between the implantable anchor and one or more tissues surrounding the implantable anchor, as well as at least one eyelet formed through the implantable anchor configured to couple at least one flexible element to the implantable anchor such that: at least a portion of the at least one flexible element may be configured to pass through a longitudinal anchor passageway that may be formed through the implantable anchor; and the at least a portion of the at least one flexible element may be configured to exit a proximal end of the implantable anchor. The anchor driver may be configured to secure the implantable anchor to the bone and may include: at least one driver channel formed in a distal portion of the anchor driver that may be configured to accommodate the at least a portion of the at least one flexible element as it passes through the longitudinal anchor passageway of the implantable anchor; and a driver shaft that may include a longitudinal shaft passageway formed therethrough that may be configured to receive the at least a portion of the at least one flexible element exiting from the proximal end of the implantable anchor to permit the at least a portion of the at least one flexible element to pass through the driver shaft of the anchor driver.

The system according to any preceding paragraph, wherein the anchor driver further includes at least one flexible element retaining mechanism disposed thereon configured to manage the at least a portion of the at least one flexible element.

The system according to any preceding paragraph, wherein the implantable anchor includes a radiolucent material.

The system according to any preceding paragraph, wherein the anchor driver further includes a driver tip disposed on a distal end of the anchor driver that may be configured to penetrate the bone and facilitate insertion of the implantable anchor into the bone.

The system according to any preceding paragraph, wherein the at least one eyelet may include a proximal eyelet formed through the implantable anchor, and a distal eyelet formed through the implantable anchor that is positioned distal to the proximal eyelet along the implantable anchor.

The system according to any preceding paragraph, wherein the anchor driver further includes a driver engagement member, and wherein the at least one driver channel may be formed in the driver engagement member.

These and other features and advantages of the present disclosure will become more fully apparent from the following description and appended claims or may be learned by the practice of the suture anchor devices, systems, and methods set forth hereinafter.

### BRIEF DESCRIPTION OF THE DRAWINGS

Exemplary embodiments of the present disclosure will become more fully apparent from the following description and appended claims, taken in conjunction with the accompanying drawings. Understanding that these drawings depict only exemplary embodiments and are, therefore, not to be considered limiting of the scope of the appended claims, the exemplary embodiments of the present disclosure will be described with additional specificity and detail through use of the accompanying drawings in which:

FIG. 1 is a perspective view of a suture anchor coupled to a suture anchor driver, according to an embodiment of the present disclosure;

FIGS. 2A-2D show detailed top, front, side, and bottom views of a driver handle, according to an embodiment of the present disclosure;

FIGS. 3A-3D show detailed top, front, side, and bottom views of a driver handle, according to another embodiment of the present disclosure;

FIGS. 4A-4D show detailed top, front, side, and bottom views of a driver handle, according to another embodiment of the present disclosure;

FIGS. 5A and 5B show side views of a standard and modified suture anchor component, according to an embodiment of the present disclosure;

FIG. 6 is a section side view of the standard suture anchor shown in FIG. 5A;

FIG. 7 is a section side view of the modified suture anchor shown in FIG. 5B;

FIG. 8 is a side view of the standard suture anchor shown in FIG. 5A coupled to the suture anchor driver of FIG. 1;

FIG. 9 is a side view of the modified suture anchor shown in FIG. 5B coupled to the suture anchor driver of FIG. 1;

FIG. 10A is a top view of the standard suture anchor shown in FIG. 5A;

FIG. 10B is a section top view of the standard suture anchor shown in FIG. 10A;

FIG. 11 is a side view of a driver engagement portion of the suture anchor driver shown in FIG. 1;

FIG. 12 is a bottom end view of the driver engagement portion shown in FIG. 11;

FIG. 13 is a perspective view of the driver engagement portion shown in FIG. 11;

FIG. 14 is a bottom end view of the standard suture anchor coupled to the suture anchor driver of FIG. 1;

FIG. 15 is a perspective view of the standard suture anchor decoupling from the suture anchor driver with sutures coupled to the suture anchor;

FIG. 16A is a section side view of the standard suture anchor coupled to the suture anchor driver prior to deployment with sutures coupled to the suture anchor;

FIG. 16B is a section side view of the standard suture anchor coupled to the suture anchor driver without sutures;

FIG. 17A is a section side view of the modified suture anchor coupled to the suture anchor driver prior to deployment with sutures coupled to the suture anchor;

FIG. 17B is a section side view of the modified suture anchor coupled to the suture anchor driver without sutures;

FIG. 18 is a side view of a driver tip, according to another embodiment of the present disclosure;

FIG. 19 is a bottom end view of the driver tip of FIG. 18; and

FIG. 20 is a perspective end view of the driver tip of FIG. 18.

It is to be understood that the drawings are for purposes of illustrating the concepts of the present disclosure and may not be drawn to scale. Furthermore, the drawings illustrate exemplary embodiments and do not represent limitations to the scope of the present disclosure.

### DETAILED DESCRIPTION

Exemplary embodiments of the present disclosure will be best understood by reference to the drawings, wherein like parts are designated by like numerals throughout. It will be readily understood that the components of the present disclosure, as generally described and illustrated in the drawings, could be arranged and designed in a wide variety of different configurations. Thus, the following more detailed description of the embodiments of the devices, systems, and methods, as represented in the drawings, is not intended to limit the scope of the present disclosure but is merely representative of exemplary embodiments of the present disclosure.

The word “exemplary” is used herein to mean “serving as an example, instance, or illustration.” Any embodiment described herein as “exemplary” is not necessarily to be construed as preferred or advantageous over other embodiments. While the various aspects of the embodiments are presented in the drawings, the drawings are not necessarily drawn to scale unless specifically indicated.

Standard medical planes of reference and descriptive terminology are employed in this specification. While these terms are commonly used to refer to the human body, certain terms may also be applicable to physical objects in general.

A standard system of three mutually perpendicular reference planes is employed. A sagittal plane divides a body into right and left portions. A coronal plane divides a body into anterior and posterior portions. A transverse plane divides a body into superior and inferior portions. A mid-sagittal, mid-coronal, or mid-transverse plane divides a body into equal portions, which may be bilaterally symmetric. The intersection of the sagittal and coronal planes defines a superior-inferior or cephalad-caudal axis. The intersection of the sagittal and transverse planes defines an anterior-posterior axis. The intersection of the coronal and transverse planes defines a medial-lateral axis. The superior-inferior or cephalad-caudal axis, the anterior-posterior axis, and the medial-lateral axis are mutually perpendicular.

Anterior means toward the front of a body. Posterior means toward the back of a body. Superior or cephalad means toward the head. Inferior or caudal means toward the feet or tail. Medial means toward the midline of a body, particularly toward a plane of bilateral symmetry of the body. Lateral means away from the midline of a body or away from a plane of bilateral symmetry of the body. Axial means toward a central axis of a body. Abaxial means away from a central axis of a body. Ipsilateral means on the same side of the body. Contralateral means on the opposite side of the body. Proximal means toward the trunk of the body. Proximal may also mean toward a user or operator. Distal means away from the trunk. Distal may also mean away from a user or operator. Dorsal means toward the top of the foot. Plantar means toward the sole of the foot. Varus means outboard deviation of the knees (away from the sagittal plane) from the line between the hip and ankle, resulting in a “bowlegged” stance. Valgus means inboard deviation of the knees (toward the sagittal plane) from the line between the hip and ankle, resulting in a “knock-kneed” stance.

It will be understood that any feature that is described or contemplated herein with respect to any device, system, method, procedure, instrument that is described or contemplated herein may be combined in any manner to produce any number of different devices, systems, methods, procedures, and/or instruments, as well as any number of different surgical kits, without departing from the spirit of scope of the present disclosure.

The present disclosure describes a suture anchor and anchor driver assembly that may be designed to enhance anchor bone purchase, ease of anchor insertion, simplify suture management, and promote bone ingrowth within the anchor. In some embodiments, a threaded, tapered suture anchor may be constructed from polyether ether ketone (PEEK), although any suitable biocompatible material may be utilized, and may be formed with one or more suture passages to allow threading of single or multiple sutures. The anchor may incorporate openings, windows, cutouts, fenestrations, or similar structures along its body to encourage bone integration, weeping of bone marrow elements to the tendon repair, and provide increased biological fixation over time.

A corresponding anchor driver may be utilized to insert and set the anchor into bone. The anchor driver may include a central lumen through which at least one suture can pass, enabling the surgeon to tension or manipulate the suture without removing the anchor driver from the anchor. In addition, the anchor driver may include at least one suture retention feature, which may comprise a cleat, tab, locking mechanism, other structure(s), etc., to secure or temporarily hold a given suture. This allows the surgeon to keep track of specific sutures (e.g., via color-coding each suture and/or organizing each sutures relative to the anchor driver). A K-wire-style threaded tip may extend through a hollow center of the anchor to enable direct puncturing of soft and or bone tissues to initiate insertion of the anchor into bone. Once the anchor is seated in the bone, the anchor driver can be disengaged and removed, leaving the anchor behind with one or more sutures secured to the anchor.

By combining a specialized anchor design with an anchor driver that facilitates suture passage and management, the assemblies of the present disclosure can reduce operating time/complexity, simplify suture handling, and enhance fixation strength for reconstructive surgeries/repairs where bone integrity may or may not be compromised.

The anchors described herein may include a tapered body with threads that may conform to the tapered body. This design can lower insertion torque, enhance fixation strength, and maximize pullout strength.

In some embodiments, the anchor may be composed of polyether ether ketone (PEEK) due to its biocompatibility and radiolucent properties. However, other materials (e.g., titanium alloys, stainless steel, bone composites, or bioabsorbable polymers) may be utilized.

In some embodiments, the anchor driver may include a penetrating or trocar tip that may project through the anchor, allowing the surgeon to puncture tissue and initiate threading in the bone directly in line with the anchor, eliminating the need pre-drill holes in the bone. This can also reduce undesirable anchor “skiving,” expansion of the prepared bone tunnel, etc., by ensuring accurate placement of the suture anchor.

In some embodiments, the suture retention mechanisms on the driver handle can enable multiple suture strands (which may be color-coded) to be distinctly organized and individually tensioned during the surgical procedure. This reduces the possibility of tangling or misidentifying suture strands during the surgical procedure, which are issues that commonly arise during the surgical procedure that require more than one suture.

While the figures and disclosure herein detail certain preferred implementations of the suture anchor system, it should be appreciated that variations in material, thread design, anchor taper, or driver-tip geometry may be introduced without departing from the spirit or scope of the present disclosure. Such modifications can be made to accommodate different bone densities, surgical approaches (e.g., arthroscopic vs. open surgical procedures), surgeon preferences, etc. The figures and disclosure herein illustrate how these various features interact and how the system may be utilized during a surgical procedure. By examining these figures, a person of skill in the art will understand the range of possible configurations and the manner in which the suture anchor and anchor driver may be further modified/adapted for use within any number of different surgical procedures.

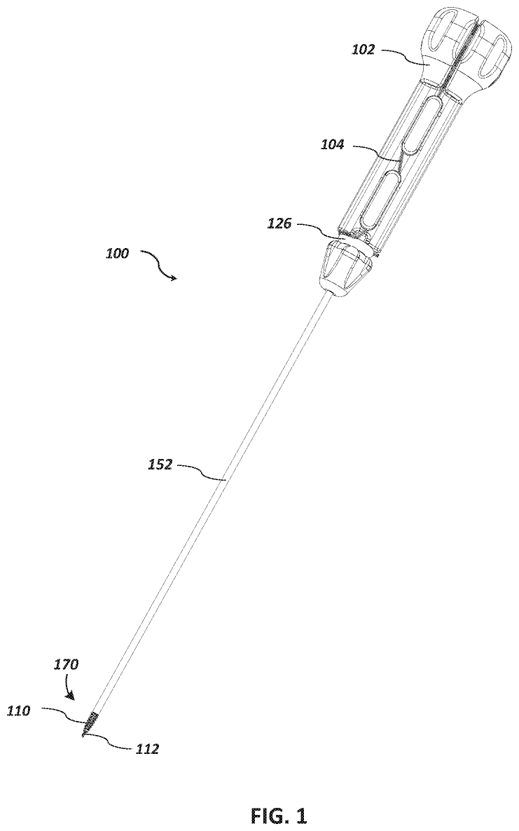

For general guidance, FIG. 1 illustrates an assembly for attaching tissue to bone that may include a suture anchor or anchor 110 coupled to a distal end of an anchor driver 100, as well as one or more sutures, surgical sutures, suture bundle 104, flexible elements, etc., that may couple with the anchor 110 and/or to the anchor driver 100, according to an embodiment of the present disclosure. The anchor driver 100 may generally include a driver handle 102 that may include an O-ring 106, a driver shaft 152 that may have a longitudinal shaft passageway 153 formed therethrough, a driver engagement member 170 disposed on a distal end of the driver shaft 152, and a driver tip 112. The driver handle 102 may be gripped by a surgeon during insertion of the anchor 110 into bone. The suture bundle 104 may contain one more sutures that may be utilized to secure tissue to bone. The O-ring 106 may provide a quick method of provisionally securing the sutures to the driver handle 102. The driver shaft 152 may extend from the driver handle 102 and couple with the anchor 110. The driver tip 112 may form the most distal end of the assembly and may be designed to engage and cut into bone tissue to provide an insertion path for the anchor 110 into the bone.

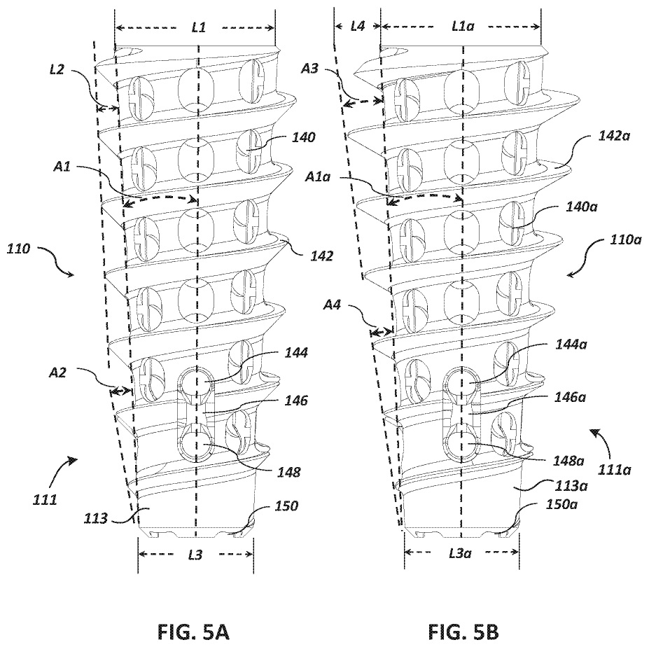

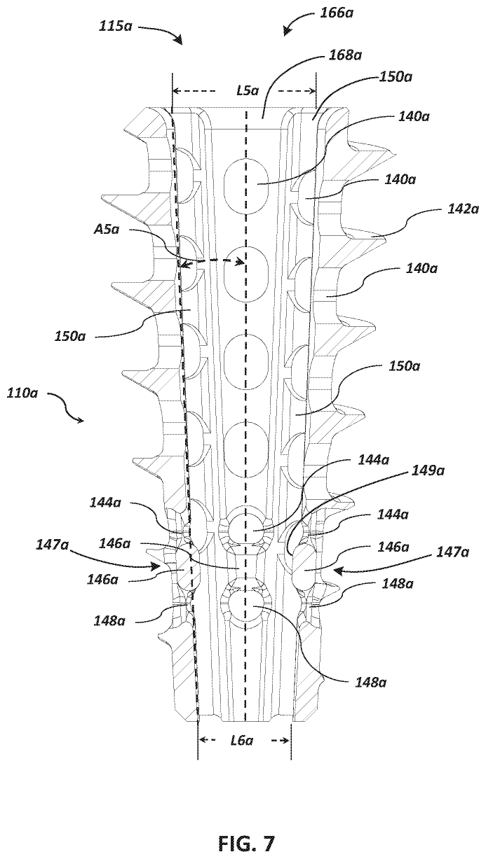

FIGS. 5A, 6, 10A, and 10B illustrate various views of the anchor 110 in more detail, and FIGS. 5B and 7 illustrate views of a slightly modified suture anchor, variant suture anchor, or anchor 110a, which may also be utilized with the anchor driver 100.

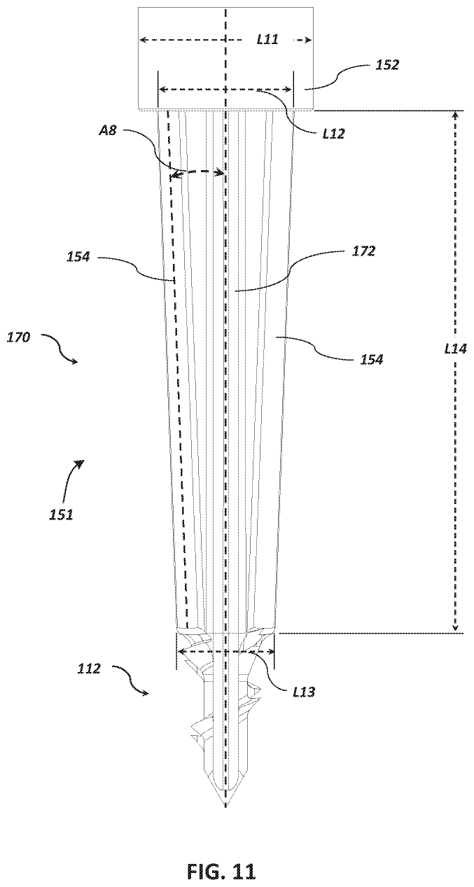

FIGS. 11-13 and 18-20 illustrate various views of a driver engagement member 170 that may be coupled with a driver tip 112 or a driver tip 112a, according to embodiments of the present disclosure.

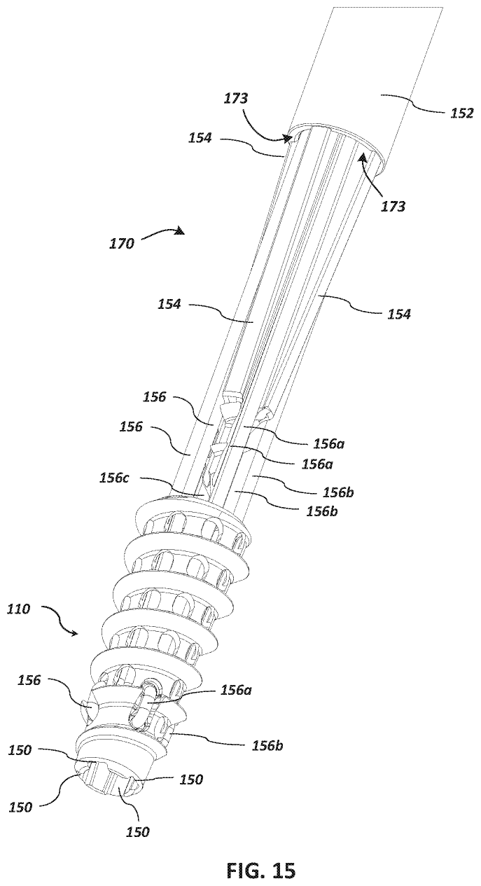

FIG. 15 illustrates the anchor 110 coupling to (or decoupling from) the driver engagement member 170 at the distal end of the anchor driver 100, including multiple sutures coupled to the anchor 110 that may pass through an interior space of the anchor 110 and into a longitudinal shaft passageway 153 that may be formed through a driver shaft 152 of the anchor driver 100, as will be discussed in more detail below.

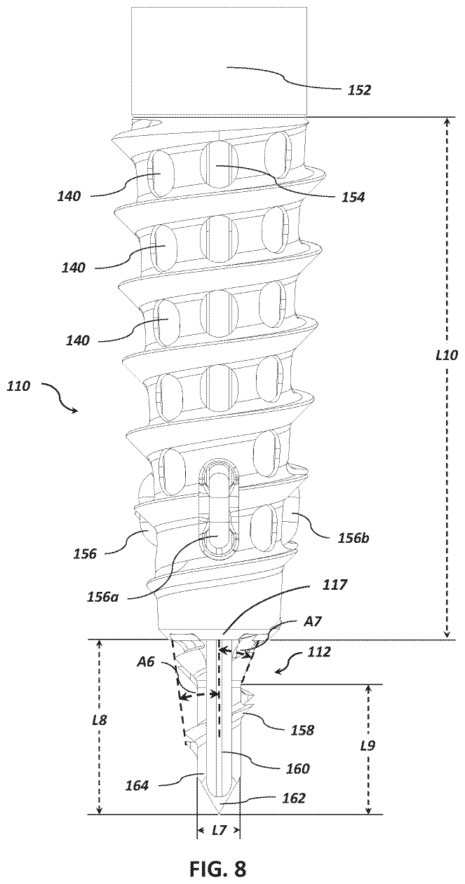

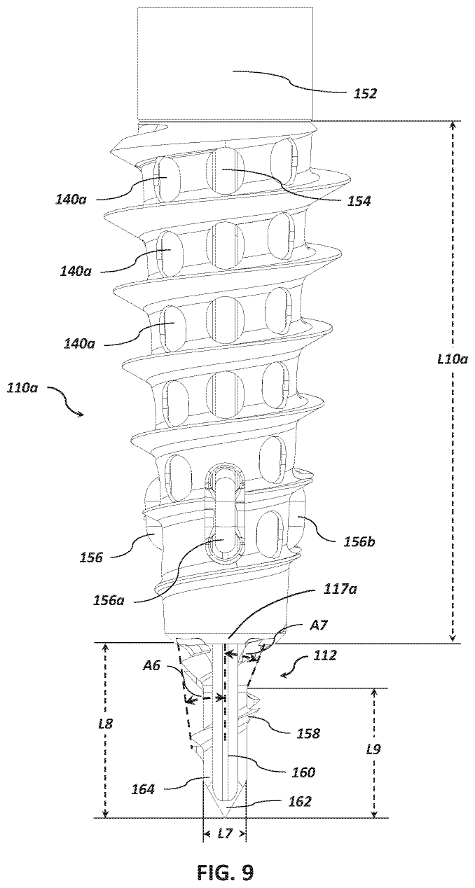

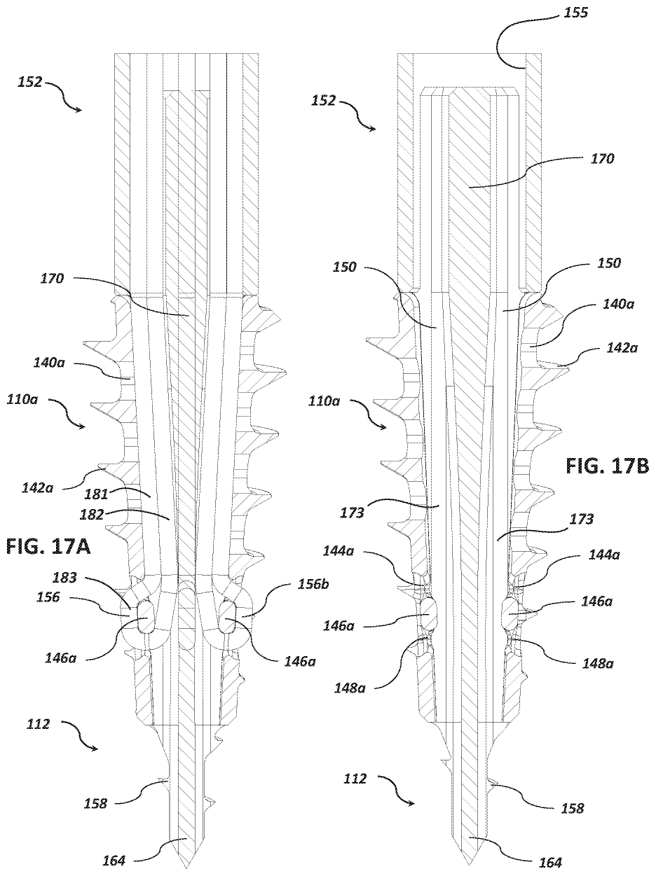

FIGS. 8, 14, 16A, 16B, and 21 illustrate various views of the anchor 110 coupled to the driver engagement member 170 of the anchor driver 100, with and without multiple sutures coupled to the anchor 110. Similarly, FIGS. 9, 17B, and 17A illustrate various views of the anchor 110a coupled to the driver engagement member 170 of the anchor driver 100, with and without multiple sutures coupled to the anchor 110a.

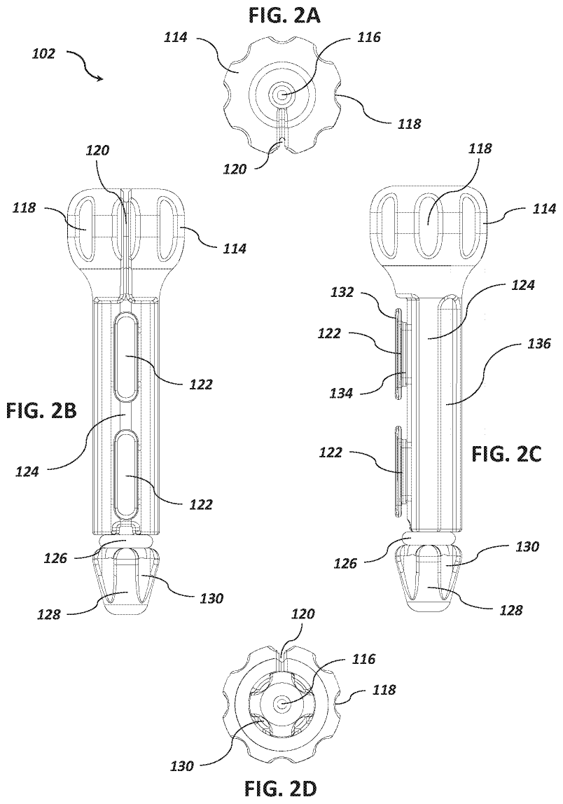

FIGS. 2A-4D illustrate various views of different driver handle embodiments that may be utilized with the anchor driver 100, as will be discussed in more detail below.

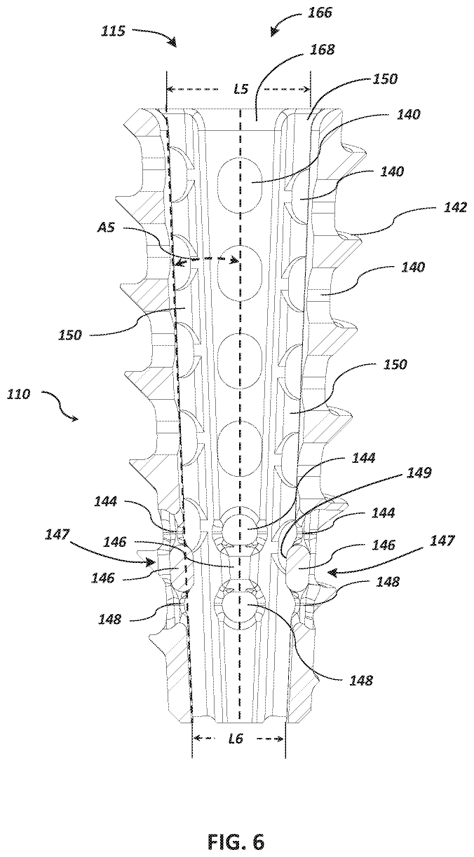

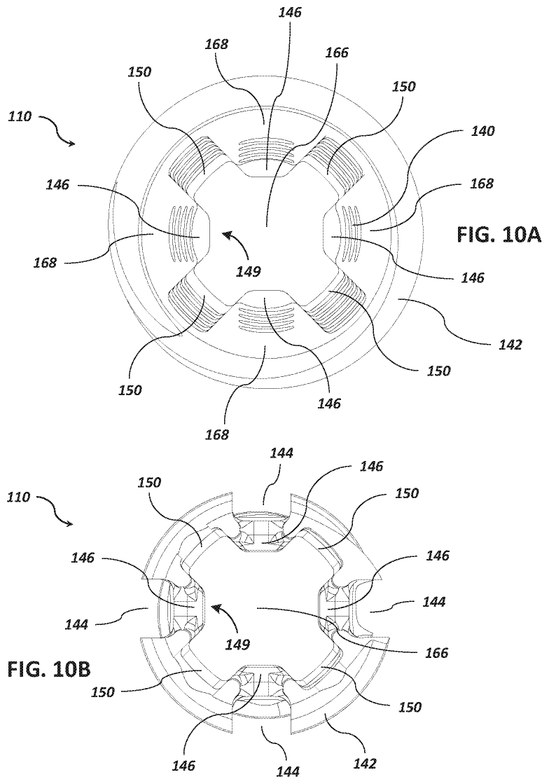

Referring to FIGS. 5A, 6, 10A, and 10B, various views of the anchor 110 are illustrated in more detail. Specifically, FIG. 5A shows a side view of the anchor 110, FIG. 6 shows a section side view of the anchor 110, FIG. 10A shows a top view of the anchor 110, and FIG. 10B shows a section top view of the anchor 110.

In some embodiments, the anchor 110 may generally include: an anchor body 111 having: a proximal end, a distal end, an outer surface 113, and an inner wall 115; a driver-receiving recess or longitudinal anchor passageway 166 formed through the anchor body 111; an anchor thread 142 disposed about the anchor body 111; at least one fenestration or fenestrations 140 formed through the anchor body 111; at least one proximal eyelet or proximal eyelet 144 formed through the anchor body 111; at least one distal eyelet or distal eyelet 148 formed through the anchor body 111 that is positioned distal to the proximal eyelet 144 along the anchor body 111; at least one bridge or bridge 146 positioned intermediate the proximal eyelet 144 and the distal eyelet 148 on the anchor body 111; at least one bridge recess or bridge recess 147 formed on the outer surface 113 of the anchor body 111 on a first side of the bridge 146; at least one bridge protrusion or bridge protrusion 149 formed on a second side of the bridge 146 or on the inner wall 115 within the anchor 110 that may transversely protrude into the longitudinal anchor passageway 166; at least one anchor slot or anchor channel 150 configured to receive a driver protrusion or driver torque connection feature 154 therein; at least one anchor rib, protrusion, or anchor torque connection feature 168 that may transversely protrude into the longitudinal anchor passageway 166 and may be configured to engage the driver torque connection feature 154 to receive a torque force transmitted from the driver torque connection feature 154 to the anchor torque connection feature 168 to rotate the anchor 110 into bone.

In some embodiments, the anchor 110 may have an outer proximal width L1 that may be greater than an outer distal width L3, due to an outer body taper angle A1 or tapered shape of the anchor body 111 along an anchor length L10 (see FIG. 8) of the anchor 110.

In some embodiments, the anchor thread 142 may have a thread taper angle A2 or tapered shape of the anchor thread 142 toward the distal end of the anchor 110.

In some embodiments, the anchor thread 142 may have a constant thread width L2 toward the proximal end of the anchor 110.

In some embodiments, the anchor 110 may have an inner proximal width L5 that may be greater than an inner distal width L6, due to an inner body taper angle A5 or tapered shape of the longitudinal anchor passageway 166 formed through the anchor 110.

In some embodiments, the anchor 110 may have one, two, three, four, or more anchor ribs, anchor channels, proximal eyelets, distal eyelets, bridges, bridge protrusions, etc.

Referring to FIGS. 5B and 7, various views of the anchor 110a are illustrated in more detail. Specifically, FIG. 5B shows a side view of the anchor 110a and FIG. 7 shows a section side view of the anchor 110a.

Analogous to the anchor 110 described above, the anchor 110a may generally include: an anchor body 111a having: a proximal end, a distal end, an outer surface 113a, and an inner wall 115a; a driver-receiving recess or longitudinal anchor passageway 166a formed through the anchor body 111a; an anchor thread 142a disposed about the anchor body 111a; at least one fenestration or fenestrations 140a formed through the anchor body 111a; at least one proximal eyelet or proximal eyelet 144a formed through the anchor body 111a; at least one distal eyelet or distal eyelet 148a formed through the anchor body 111a that may be positioned distal to the proximal eyelet 144a along the anchor body 111a; at least one bridge or bridge 146a positioned intermediate the proximal eyelet 144a and the distal eyelet 148a on the anchor body 111a; at least one bridge recess or bridge recess 147a formed on the outer surface 113a of the anchor body 111a on a first side of the bridge 146a; at least one bridge protrusion or bridge protrusion 149a formed on a second side of the bridge 146a or on the inner wall 115a within the anchor 110a that may transversely protrude into the longitudinal anchor passageway 166a; at least one anchor slot or anchor channel 150a configured to receive a driver protrusion or driver torque connection feature 154 therein; at least one anchor rib or anchor torque connection feature 168a that may transversely protrude into the longitudinal anchor passageway 166a and may be configured to engage the driver torque connection feature 154 to receive a torque force transmitted from the driver torque connection feature 154 to the anchor torque connection feature 168a to rotate the anchor 110a into bone.

In some embodiments, the anchor 110a may have an outer proximal width L1a that may be greater than an outer distal width L3a, due to an outer body taper angle A1a or tapered shape of the anchor body 111a along an anchor length L10a (see FIG. 9) of the anchor 110a.

In some embodiments, the anchor thread 142a may have a first or distal thread taper angle A4 as the anchor thread 142a tapers toward the distal end of the anchor 110a.

In some embodiments, the anchor thread 142a may have a second or proximal thread taper angle A3 as the anchor thread 142a tapers toward the proximal end of the anchor 110a.

In some embodiments, the anchor thread 142a may have a proximal thread width L4 toward the proximal end of the anchor 110a.

In some embodiments, the anchor 110a may have an inner proximal width L5a that may be greater than an inner distal width L6a, due to an inner body taper angle A5a or tapered shape of the longitudinal anchor passageway 166a formed through the anchor 110a.

In some embodiments, the anchor 110a may have one, two, three, four, or more anchor ribs, anchor channels, proximal eyelets, distal eyelets, bridges, bridge protrusions, etc.

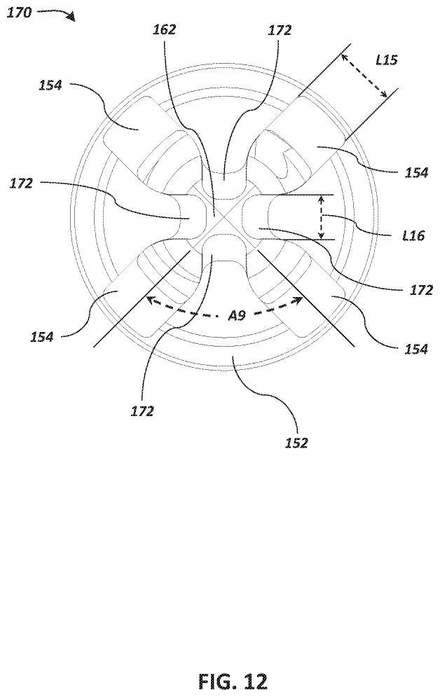

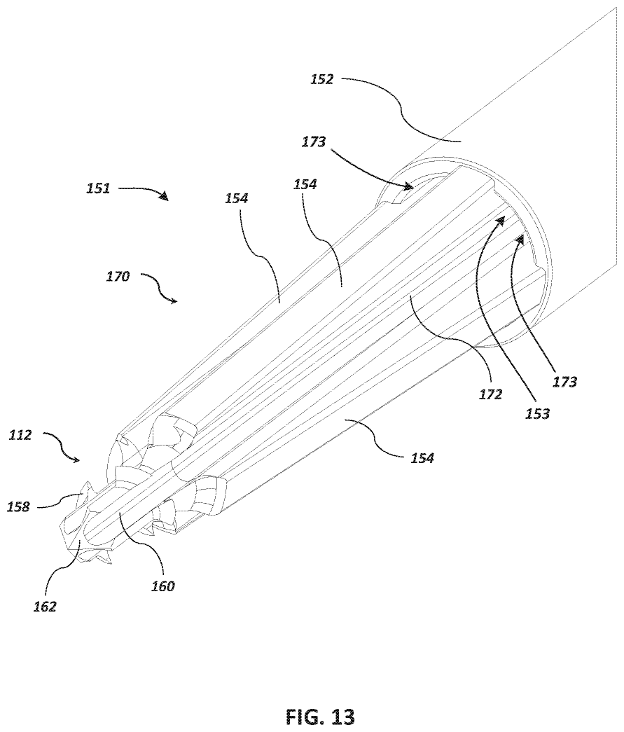

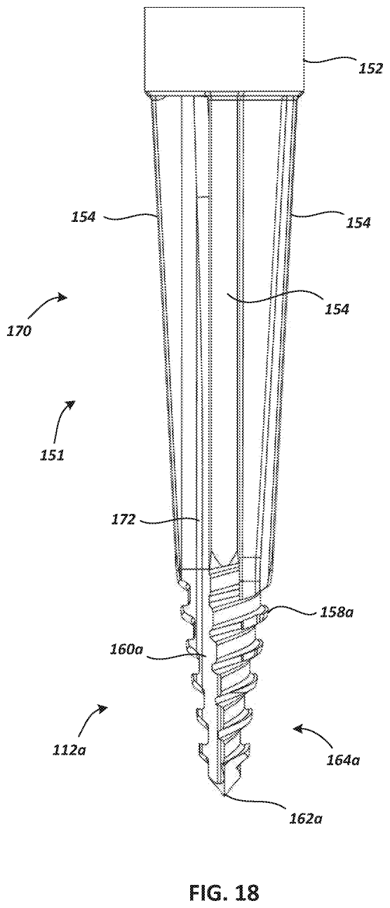

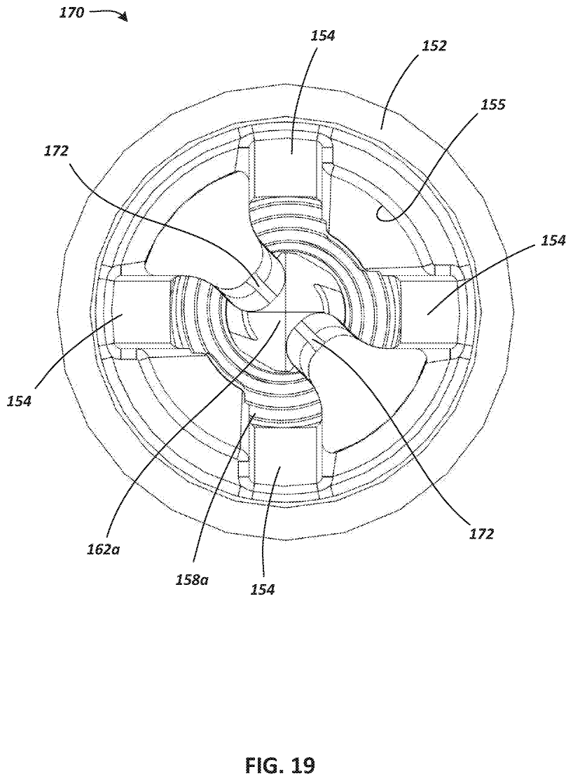

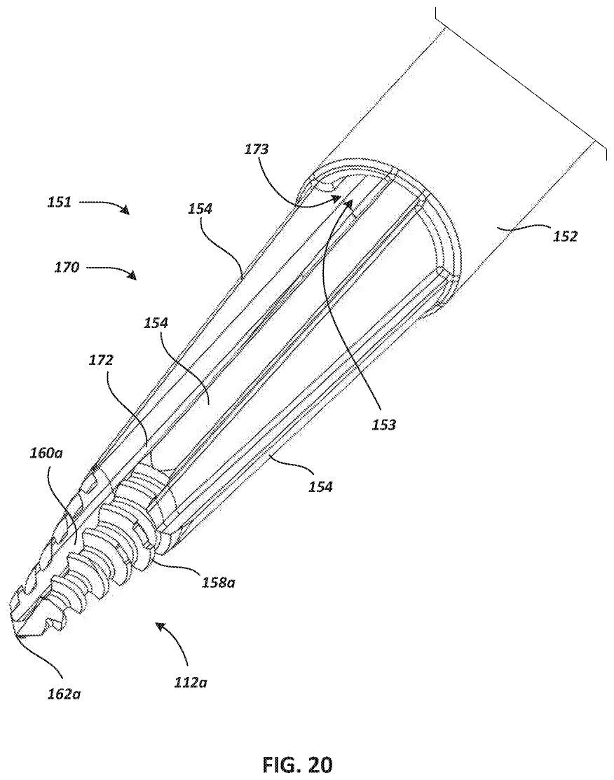

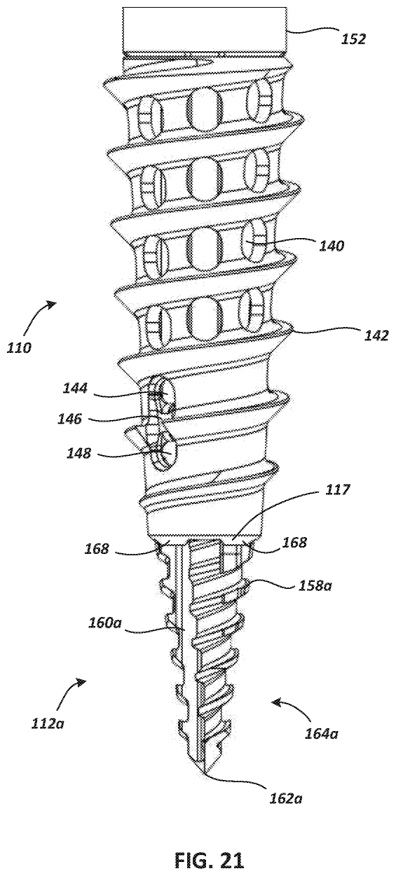

Referring to FIGS. 11-13 and 18-20, various views of the driver engagement member 170, coupled with either the driver tip 112 (see FIGS. 11-13) or the driver tip 112a (see FIGS. 18-20), are illustrated in more detail. Specifically, FIGS. 11 and 18 show side views of the driver engagement member 170, FIGS. 12 and 19 show bottom end views of the driver engagement member 170, and FIGS. 13 and 20 show perspective views of the driver engagement member 170.

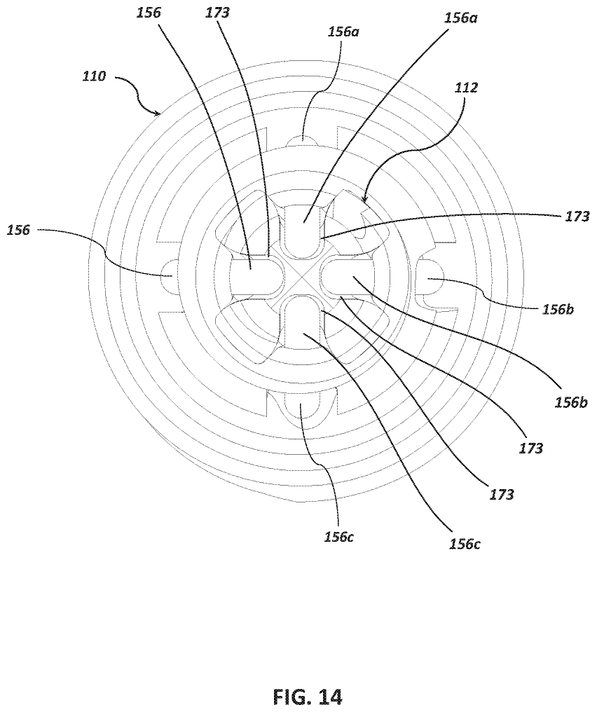

In some embodiments, the driver engagement member 170 may generally include: an elongate engagement body 151 having a tapered shape, a proximal end, a distal end, and a longitudinal axis; at least one protrusion, rib, or driver torque connection feature 154 that may project laterally away from the elongate engagement body 151 and/or be shaped for reception within the anchor channel (150, 150a) via sliding engagement therewith during insertion; and at least one recess, slot, or driver channel 172 formed in the elongate engagement body 151 that may be shaped to at least partially receive the anchor torque connection feature (168, 168a) therein via sliding engagement during insertion, while also leaving an interior space 173 between the inner wall (115, 115a) or anchor torque connection feature (168, 168a) of the anchor (110, 110a) and the driver channel 172 of the driver engagement member 170 for allowing a suture to pass through the anchor (110, 110a) along the interior space 173 that is formed therebetween (e.g., see FIGS. 14-17B).

In some embodiments, the proximal end of the driver engagement member 170 may be coupled to and/or within the distal end of the driver shaft 152, as shown in FIGS. 11, 13, and 16A-20.

In some embodiments, the interior space 173 or interior space(s) that may be formed between the inner wall (115, 115a) or anchor torque connection feature (168, 168a) of the anchor (110, 110a) and the driver channel(s) 172 of the driver engagement member 170 may extend at least partially into the distal end of the longitudinal shaft passageway 153 and be formed between an inner driver shaft wall 155 and the driver channel(s) 172 (where the driver engagement member 170 couples within the longitudinal shaft passageway 153 of the driver shaft 152, as shown in FIGS. 13, 16A-17B, and 20). In this manner, the interior spaces 173 that may be formed between the anchor (110, 110a) and the driver channel(s) 172 can continue to extend between the inner driver shaft wall 155 and the driver channel(s) 172 within the distal end of the driver shaft 152 and allow the suture(s) to pass through the interior of the anchor (110, 110a) along these interior spaces 173, exit out of the distal end of the anchor (110, 110a), enter into the interior space(s) 173 formed within the distal end of the driver shaft 152, thereby allowing the suture(s) to continue traveling through the longitudinal shaft passageway 153 of the driver shaft 152 to eventually exit from the proximal end of the driver shaft 152.

In some embodiments, the driver engagement member 170 may have a driver engagement member length L14 (see FIG. 11).

In some embodiments, the longitudinal shaft passageway 153 that is formed through the driver shaft 152 may have a driver shaft width L11.

In some embodiments, the driver engagement member 170 may have a proximal driver engagement member width L12 that may be greater than a distal driver engagement member width L13 of the driver engagement member 170, due to is tapered shape.

In some embodiments, the driver engagement member 170 may have a driver rib angle A8, due to the tapered shapes of the driver ribs.

In some embodiments, the driver engagement member 170 may have a driver rib width L15, a driver recess width L16, and a driver rib angle A9, as seen in FIG. 12.

In some embodiments, the driver engagement member 170 may have one, two, three, four, or more driver ribs and/or driver channels.

In some embodiments, a distal end of the driver engagement member 170 may couple with a proximal end of the driver tip 112 (e.g., see FIGS. 11-13) and/or the proximal end of the driver tip 112a (e.g., see FIGS. 18-20).

In some embodiments, all or at least a portion of the driver tip (112, 112a) may be configured to protrude from the distal end of the anchor (110, 110a) while the anchor (110, 110a) is coupled to the driver engagement member 170 (e.g., see FIGS. 8, 9, 16A-17B, and 21). In this manner, the driver tip (112, 112a) can puncture/penetrate the bone first to provide an initial pathway for the anchor (110, 110a) into the bone. Advantageously, the driver tip (112, 112a) can be made of a strong material (e.g., metal) to perform this puncture/penetrate function, thereby reducing the mechanical stresses imparted to the anchor (110, 110a) during insertion and/or allowing the anchor (110, 110a) to be made from relatively weaker materials than the driver tip (112, 112a). Moreover, the driver tip (112, 112a) is configured to be removed from the bone after the anchor (110, 110a) is inserted into the bone by retracting the anchor driver 100 from the anchor (110, 110a). This may advantageously remove any metal materials from the bone (e.g., in cases where the driver tip is made of metal materials), which may be radiopaque to x-rays and/or have other undesirable characteristics for long term implantation within the patient. In some embodiments, the anchor (110, 110a) may be formed of a radiolucent material. This can eliminate any possible imaging artifacts from metal materials that may interfere with x-ray, MRI, CT scans, etc. In contrast, some conventional suture anchor systems may incorporate metal tips within the anchor body that remain after deployment, creating unwanted imaging artifacts that can interfere with subsequent medical imaging techniques.

In the embodiment shown in FIGS. 8, 9, 11-13, and 16A-16B, the driver tip 112 may generally include: a driver tip shank 164, a tip or trocar tip 162 disposed on a distal end of the driver tip shank 164, a driver tip thread 158 disposed about the driver tip shank 164, and a driver tip channel 160 or channels formed in the driver tip shank 164.

In some embodiments, the driver tip channels 160 may be aligned with the driver channels 172 of the driver engagement member 170.

In some embodiments, the driver tip 112 may have a driver tip length or an exposed driver tip length L8 (see FIGS. 9 and 11).

In some embodiments, the driver tip shank 164 may have a distal driver tip length L9 with a driver distal tip width L7 that may be constant along at least a portion of the driver tip 112, which may then be configured to transition to a tapered portion near the distal end of the anchor (110, 110a) to match, or substantially match, a tapered angle of a chamfered edge (117, 117a) that may be formed on the distal end of the anchor (110, 110a) to help facilitate insertion of the anchor (110, 110a) into bone.

In some embodiments, the driver tip 112 may have a driver tip tapered portion angle A7 and/or a driver tip thread angle A6, as shown in FIGS. 8 and 9.

In the embodiment shown in FIGS. 18-21, the driver tip 112a may generally include: a driver tip shank 164a, a tip or trocar tip 162a disposed on a distal end of the driver tip shank 164a, a driver tip thread 158a disposed about the driver tip shank 164a, and a driver tip channel 160a or channels formed in the driver tip shank 164a.

In some embodiments, the driver tip channel 160a or channels may be aligned with the driver channels 172 of the driver engagement member 170.

In some embodiments, the driver tip shank 164a may be configured to taper and match, or substantially match, a tapered angle of a chamfered edge (117, 117a) that may be formed on the distal end of the anchor (110, 110a) to help facilitate insertion of the anchor (110, 110a) into bone.

FIG. 15 illustrates the anchor 110 being coupled to (and/or decoupling from) the driver engagement member 170 that is disposed at the distal end of the anchor driver 100 with four sutures or flexible elements coupled to the anchor 110 at four bridges of the anchor 110 including: a first flexible element 156, a second flexible element 156a, a third flexible element 156b, and a fourth flexible element 156c. Each of these flexible elements are configured to pass through the interior of the anchor 110 along interior spaces 173 that may be formed between the inner wall of the anchor 110 and the driver channels 172 of the driver engagement member 170 (as previously discussed), and then continue along the interior spaces 173 that may extend into the distal end of the driver shaft 152. In this manner, the flexible elements may continue through the longitudinal shaft passageway 153 and exit from the proximal end of the driver shaft 152.

FIGS. 8, 14, 16A, 16B, and 21 illustrate various views of the anchor 110 coupled to the driver engagement member 170 of the anchor driver 100, both with and without multiple sutures coupled to the anchor 110. Similarly, FIGS. 9, 17B, and 17A illustrate various views of the anchor 110a coupled to the driver engagement member 170 of the anchor driver 100, both with and without multiple sutures coupled to the anchor 110a.

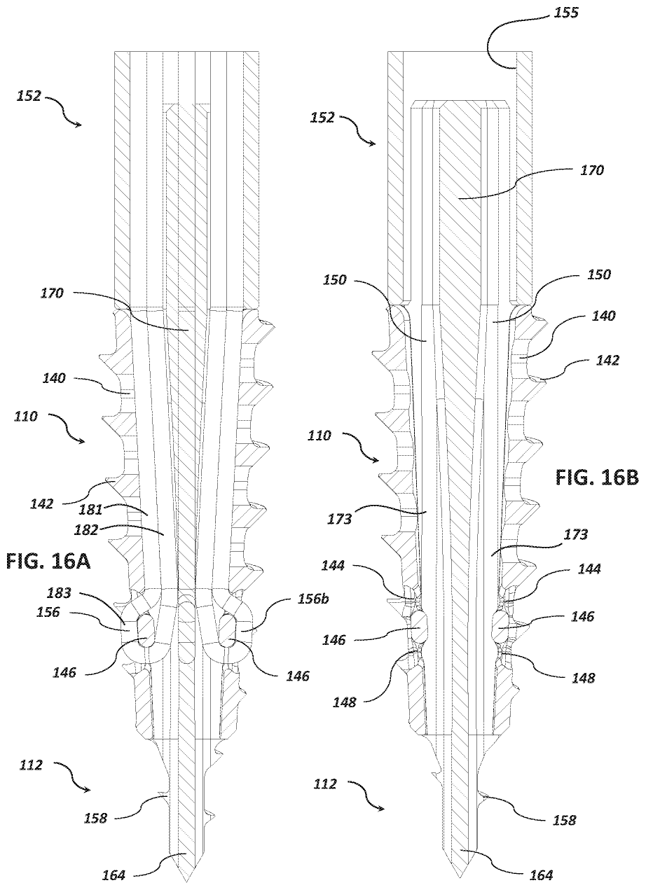

As shown in FIGS. 16A and 16B, a first portion 181 and/or a second portion 182 of the flexible element 156 may be configured to pass through the proximal eyelet and the distal eyelet to couple the flexible element 156 to the bridge 146 at a third portion 183 of the flexible element that is engaged with the bridge 146. The first portion 181 and the second portion 182 of the flexible element 156 may then pass through the anchor (110, 110a) along an interior space that is formed between the inner wall of the anchor (110, 110a) and a driver channel 172, as previously described herein.

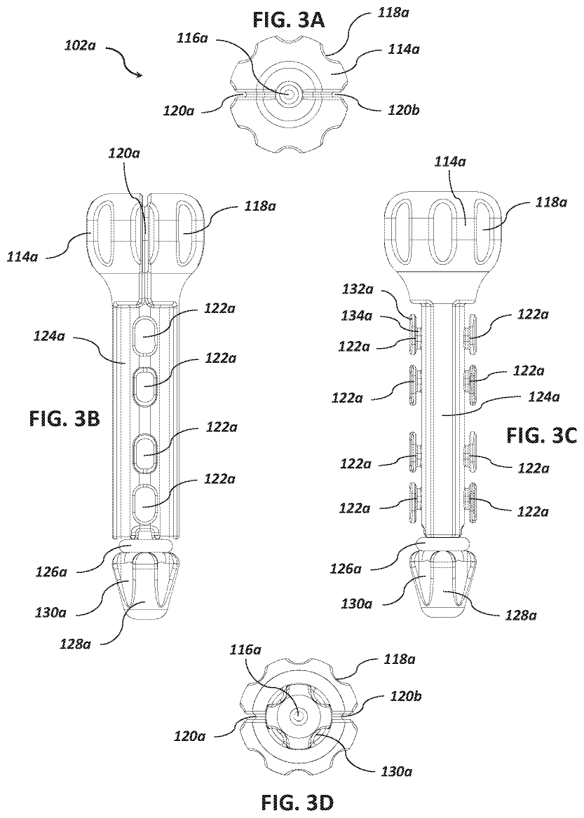

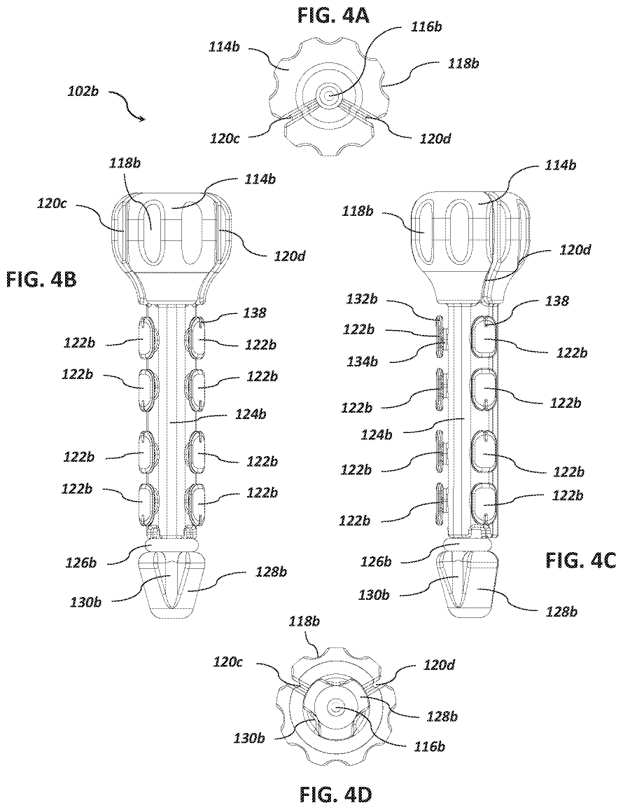

FIGS. 2A-4D illustrate various views of different driver handle embodiments that may be utilized with the anchor driver 100, according to embodiments of the present disclosure. Specifically, FIGS. 2A-2D show detailed top, front, side, and bottom views of a first driver handle embodiment, FIGS. 3A-3D show detailed top, front, side, and bottom views of a second driver handle embodiment, and FIGS. 4A-4D show detailed top, front, side, and bottom views of a third driver handle embodiment. However, it will be understood that the various features of these different driver handle embodiments can be mixed and matched with each other in any combination to form any number of different driver handle embodiments, without departing from the spirit or scope of the present disclosure. In this manner, the various features of these different driver handle embodiments can be adapted to organize/arrange/manage any number of different flexible elements or sutures to facilitate any number of different surgical procedures.

In some embodiments, the driver handles (102, 102a, 102b) may generally include: a handle body (124, 124a, 124b) having a proximal end, a distal end, and a longitudinal axis; one or more handle body scallops 136 that may be formed in the handle body (124, 124a, 124b); a longitudinal handle passageway (116, 116b, 116c) that may be formed through the driver handles (102, 102a, 102b); a knob or palm grip (114, 114a, 114b) that may be disposed on the proximal end of the handle body (124, 124a, 124b); one or more palm grip scallops (118, 118a, 118b) that may be formed in the palm grip (114, 114a, 114b); one or more slots (e.g., a first slot 120, a second slot 120a, a third slot 120b, a fourth slot 120c, a fifth slot 120d, etc.) that may be formed in the palm grip (114, 114a, 114b); a handle tip (128, 128a, 128b) that may be disposed on the distal end of the handle body (124, 124a, 124b); one or more handle tip scallops (130, 130a, 130b) that may be formed in the handle tip (128, 128a, 128b); an O-ring (126, 126a, 126b) that may be positioned intermediate the handle tip (128, 128a, 128b) and the handle body (124, 124a, 124b); one or more flexible element retaining mechanisms or suture cleats (122, 122a, 122b) that may be positioned along the handle body (124, 124a, 124b) and may include one or more suture cleat necks (134, 134a, 134b) and one or more suture cleat heads (132, 132a, 132b); and one or more locking mechanisms 138 formed in the one or more suture cleats (122, 122a, 122b) which may be configured to grasp and lock one or more flexible elements or sutures to the one or more suture cleats (122, 122a, 122b).

In some embodiments, the one or more slots (120, 120a, 120b, 120c, 120d) may be configured to receive one or more flexible elements therein to guide the one or more flexible elements along the one or more transverse orientations of the one or more slots (120, 120a, 120b, 120c, 120d) relative to the proximal end of the driver handle (102, 102a, 102b) toward at least one of the one or more suture cleats (122, 122a, 122b) which may be disposed on a side of the driver handle (102, 102a, 102b).

In some embodiments, the one or more slots (120, 120a, 120b, 120c, 120d) may include a plurality of slots formed in the proximal end of the driver handle (102, 102a, 102b) that may be configured to receive a plurality of flexible elements therein to guide the plurality of flexible elements along the plurality of transverse orientations of the one or more slots (120, 120a, 120b, 120c, 120d) relative to the proximal end of the driver handle (102, 102a, 102b) toward at least one of the one or more suture cleats (122, 122a, 122b) which may be disposed on a side of the driver handle (102, 102a, 102b).

In some embodiments, the one or more flexible element retaining mechanisms or suture cleats (122, 122a, 122b) may include a first plurality of flexible element retaining mechanisms disposed on a first side of the driver handle, and a second plurality of flexible element retaining mechanisms disposed on a second side of the driver handle.

In some embodiments, the O-ring (126, 126a, 126b) may be configured to secure one or more flexible elements to the driver handle (102, 102a, 102b) by wedging and trapping the one or more flexible elements between the O-ring (126, 126a, 126b) and a surface of the driver handle (102, 102a, 102b).

In some embodiments, the one or more flexible elements may be configured to pass through the longitudinal shaft passageway 153, exit from the proximal end of the driver shaft 152, enter into the longitudinal handle passageway (116, 116b, 116c), and exit from the proximal end of the driver handle (102, 102a, 102b).

In operation, the suture anchor and anchor driver may functions as follows for at least some embodiments: during initial assembly, the flexible elements (156, 156a, 156b, 156c) may be threaded through the longitudinal handle passageway (116, 116a, 116b) of the driver handle (102, 102a, 102b), through the longitudinal shaft passageway 153 of the driver shaft (152), along the driver channels (172), through one of the distal eyelets (148, 148a) or the proximal eyelets (144, 144a) of the anchor (110, 110a), around the bridges (146, 146a), back through the other one of the distal eyelets (148, 148a) and the proximal eyelets (144, 144a), back along the driver channels (172), through the longitudinal shaft passageway 153 of the driver shaft (152), through the longitudinal handle passageway (116, 116a, 116b) of the driver handle (102, 102a, 102b), along the slots (120, 120a, 120b, 120c, 120d) and secured to one or more of the suture cleats (122, 122a, 122b) on the driver handle (102, 102a, 102b).

This procedure can be reversed during assembly where the flexible element (156, 156a, 156b, 156c) may be threaded first through one of the distal eyelets (148, 148a) or the proximal eyelets (144, 144a) of the anchor (110, 110a), around the bridges (146, 146a), and back through the other one of the distal eyelets (148, 148a) and the proximal eyelets (144, 144a). The two ends of the flexible element (156, 156a, 156b, 156c) may then be fed along the driver channels (172) that may be placed within the anchor (110, 110a), into the longitudinal shaft passageway (153) of the driver shaft (152), through the longitudinal handle passageway (116, 116a, 116b) of the driver handle (102, 102a, 102b), along the slots (120, 120a, 120b, 120c, 120d) and secured to one or more of the suture cleats (122, 122a, 122b) on the driver handle (102, 102a, 102b).

The surgeon may then position the assembled driver and anchor at a desired location on a bone, and the trocar tip (162, 162a) may then be utilized to initiate penetration into the bone. The surgeon may then apply a rotational force to the driver handle (102, 102a, 102b) to rotate the anchor (110, 110a) into the bone. The anchor thread (142, 142a) may engage the bone and provide secure fixation of the anchor (110, 110a) within the bone. Once the anchor (110, 110a) is seated in the bone, the anchor driver 100 may be withdrawn, leaving the anchor (110, 110a) and attached sutures or flexible elements in place. The surgeon may then utilize these sutures to secure one or more tissues or soft tissues to the bone as desired for the surgical procedure.

Any procedures or methods disclosed herein may comprise one or more steps or actions for performing the described method. The method steps and/or actions may be interchanged with one another. In other words, unless a specific order of steps or actions is required for proper operation of the embodiment, the order and/or use of specific steps and/or actions may be modified.

Reference throughout this specification to “an embodiment” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

Similarly, it should be appreciated that in the above description of embodiments, various features are sometimes grouped together in a single embodiment, figure, or description thereof for the purpose of streamlining the present disclosure. This method of disclosure, however, is not to be interpreted as reflecting an intention that any embodiment requires more features than those expressly recited in that embodiment. Rather, inventive aspects lie in a combination of fewer than all features of any single foregoing disclosed embodiment.

Recitation of the term “first” with respect to a feature or element does not necessarily imply the existence of a second or additional such feature or element. Elements recited in means-plus-function format are intended to be construed in accordance with 35 U.S.C. § 112. It will be apparent to those having skill in the art that changes may be made to the details of the above-described embodiments without departing from the underlying principles set forth herein.

The phrases “connected to”, “coupled to”, “engaged with”, and “in communication with” may refer to any form of interaction between two or more entities, including mechanical, electrical, magnetic, electromagnetic, fluid, and thermal interaction. Two components may be functionally coupled to each other even though they are not in direct contact with each other. The term “coupled” can include components that are coupled to each other via integral formation (e.g., integrally formed as a single piece, etc.), components that are removably and/or non-removably coupled with each other, components that are functionally coupled to each other through one or more intermediary components, etc. The term “abutting” refers to items that may be in direct physical contact with each other, although the items may not necessarily be attached together. The phrase “fluid communication” refers to two or more features that are connected such that a fluid within one feature is able to pass into another feature.

As defined herein the term “substantially” means within +/−20% of a target value, measurement, or desired characteristic.

The use of “e.g.,” and “or” indicates non-exclusive alternatives without limitation, unless otherwise noted. The terms “attached,” “coupled,” and grammatically related terms refer to the fixed, releasable, or integrated association of two or more elements with or without one or more other elements in between. Thus, the terms “attached” or “coupled” include releasably attaching or fixedly attaching two or more elements in the presence or absence of one or more other elements in between.

As utilized herein with respect to structural features (e.g., to describe shape, size, orientation, direction, relative position, etc.), the terms “approximately,” “about,” “substantially,” and similar terms are meant to cover minor variations in structure that may result from, for example, the manufacturing or assembly process and are intended to have a broad meaning in harmony with the common and accepted usage by those of ordinary skill in the art to which the subject matter of this disclosure pertains. Accordingly, these terms should be interpreted as indicating that insubstantial or inconsequential modifications or alterations of the subject matter described and claimed are considered to be within the scope of the disclosure as recited in the appended claims.

It should be noted that the term “exemplary” and variations thereof, as used herein to describe various embodiments, are intended to indicate that such embodiments are possible examples, representations, or illustrations of possible embodiments (and such terms are not intended to connote that such embodiments are necessarily extraordinary or superlative examples).

The term “coupled” and variations thereof, as used herein, means the joining of two members directly or indirectly to one another. Such joining may be stationary (e.g., permanent or fixed) or moveable (e.g., removable or releasable). Such joining may be achieved with the two members coupled directly to each other, with the two members coupled to each other using a separate intervening member and any additional intermediate members coupled with one another, or with the two members coupled to each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above.

References herein to the positions of elements (e.g., “top,” “bottom,” “above,” “below”) are merely used to describe the orientation of various elements in the FIGURES. It should be noted that the orientation of various elements may differ according to other exemplary embodiments, and that such variations are intended to be encompassed by the present disclosure.

Although the figures and description may illustrate a specific order of method steps, the order of such steps may differ from what is depicted and described, unless specified differently above. Also, two or more steps may be performed concurrently or with partial concurrence, unless specified differently above.

It is important to note that any element disclosed in one embodiment may be incorporated or utilized with any other embodiment disclosed herein. The devices, systems and methods described herein may be embodied in other specific forms without departing from the characteristics thereof. The foregoing implementations are illustrative rather than limiting of the described systems and methods. The scope of the systems and methods described herein is thus indicated by the appended claims, rather than the foregoing description, and changes that come within the meaning and range of equivalency of the claims are embraced therein.

While specific embodiments and applications of the present disclosure have been illustrated and described, it is to be understood that the scope of the present disclosure is not limited to the precise configurations and components disclosed herein. Various modifications, changes, and variations which will be apparent to those skilled in the art may be made in the arrangement, operation, and details of the devices, systems, and methods disclosed herein.

What is claimed is:

1. An assembly for attaching tissue to bone comprising:
an anchor driver comprising:
a driver shaft comprising:
a proximal end;
a distal end;
a longitudinal axis; and
a longitudinal shaft passageway formed through the driver shaft between the proximal end and the distal end of the driver shaft;
a driver engagement member proximate the distal end of the driver shaft, the driver engagement member comprising:
a driver torque connection feature; and
a driver channel; and
a driver tip at a distal end of the driver engagement member;
an anchor configured to removably couple with the driver engagement member, the anchor comprising:
a proximal end;
a distal end;
a longitudinal axis;
a longitudinal anchor passageway formed through the anchor between the proximal end and the distal end of the anchor defining an inner wall within the anchor; and
an anchor torque connection feature configured to engage with the driver torque connection feature and receive a torque force transmitted therefrom; and
a flexible element configured to couple with the anchor;
wherein, when the anchor is coupled to the driver engagement member of the anchor driver, and the flexible element is coupled to the anchor:
the driver tip is configured to protrude from the distal end of the anchor;
the flexible element is configured to pass through the anchor and into the longitudinal shaft passageway along an interior space that is formed between the inner wall of the anchor and the driver channel; and
the flexible element is configured to pass through the longitudinal shaft passageway and exit the proximal end of the driver shaft.

2. The assembly of claim 1, wherein:
the driver torque connection feature comprises a plurality of driver torque connection features;
the anchor torque connection feature comprises a plurality of anchor torque connection features configured to engage with the plurality of driver torque connection features and receive the torque force transmitted therefrom;
the driver channel comprises a plurality of driver channels;
the flexible element comprises a plurality of flexible elements configured to couple with the anchor;
the plurality of flexible elements are configured to pass through the anchor and into the longitudinal shaft passageway along a plurality of interior spaces that are formed between the inner wall of the anchor and the plurality of driver channels; and
the plurality of flexible elements are configured to pass through the longitudinal shaft passageway and exit the proximal end of the driver shaft.

3. The assembly of claim 1, wherein:
the driver tip comprises:
a driver tip shank; and
at least one driver tip channel formed in the driver tip shank.

4. The assembly of claim 3, wherein the driver tip shank comprises at least one driver tip thread disposed about the driver tip shank.

5. The assembly of claim 3, wherein at least a portion of the driver tip shank comprises a constant width.

6. The assembly of claim 3, wherein the driver tip shank comprises a tapered shape.

7. The assembly of claim 1, wherein the anchor comprises at least one bridge configured to couple the flexible element to the anchor.

8. An assembly for attaching tissue to bone comprising:
a driver handle comprising:
a proximal end;
a distal end;
a longitudinal axis; and
a longitudinal handle passageway formed through the driver handle between the proximal end and the distal end of the driver handle;
a driver shaft comprising:
a proximal end coupled to or integrated with the distal end of the driver handle;
a distal end;
a longitudinal axis; and
a longitudinal shaft passageway formed through the driver shaft between the proximal end and the distal end of the driver shaft;
a driver engagement member proximate the distal end of the driver shaft, the driver engagement member comprising:
a driver torque connection feature; and
a driver channel; and
a driver tip at a distal end of the driver engagement member;
wherein:
the driver tip is configured to protrude from a distal end of an anchor that is coupled with the driver engagement member;
a flexible element coupled to the anchor is configured to pass through the anchor and into the longitudinal shaft passageway along an interior space that is formed between an inner wall of the anchor and the driver channel;
the flexible element is configured to pass through the longitudinal shaft passageway and exit the proximal end of the driver shaft; and
the flexible element is configured to pass through the longitudinal handle passageway and exit the proximal end of the driver handle.

9. The assembly of claim 8, wherein:
the driver torque connection feature comprises a plurality of driver torque connection features configured to engage a plurality of anchor torque connection features formed within the anchor and transmit a torque force to the anchor;
the driver channel comprises a plurality of driver channels;
the flexible element comprises a plurality of flexible elements coupled to the anchor;
the plurality of flexible elements are configured to pass through the anchor and into the longitudinal shaft passageway along a plurality of interior spaces that are formed between the inner wall of the anchor and the plurality of driver channels;
the plurality of flexible elements are configured to pass through the longitudinal shaft passageway and exit the proximal end of the driver shaft; and
the plurality of flexible elements are configured to pass through the longitudinal handle passageway and exit the proximal end of the driver handle.

10. The assembly of claim 8, wherein the proximal end of the driver handle comprises a slot formed therein that is configured to receive the flexible element therein and guide the flexible element along a transverse orientation relative to the proximal end of the driver handle.

11. The assembly of claim 10, wherein the slot comprises a plurality of slots formed in the proximal end of the driver handle that are configured to receive a plurality of flexible elements therein to guide the plurality of flexible elements along a plurality of transverse orientations relative to the proximal end of the driver handle.

12. The assembly of claim 8, wherein the distal end of the driver handle comprises an O-ring configured to secure the flexible element to the driver handle.

13. The assembly of claim 8, wherein the driver handle comprises a flexible element retaining mechanism disposed on a first side of the driver handle.

14. The assembly of claim 13, wherein:
the flexible element retaining mechanism comprises a first plurality of flexible element retaining mechanisms disposed on the first side of the driver handle; and
the driver handle comprises a second plurality of flexible element retaining mechanisms disposed on a second side of the driver handle.

15. The assembly of claim 13, wherein the flexible element retaining mechanism comprises a locking mechanism configured to grasp and lock the flexible element to the flexible element retaining mechanism.

16. An assembly for attaching tissue to bone comprising:
a flexible element comprising:
a first portion;
a second portion; and
a third portion; and
an anchor comprising:
an anchor body;
a proximal end;
a distal end;
a longitudinal axis;
a longitudinal anchor passageway formed through the anchor between the proximal end and the distal end of the anchor defining an inner wall within the anchor;
an anchor torque connection feature formed within the anchor;
a proximal eyelet formed through the anchor;
a distal eyelet formed through the anchor distal to the proximal eyelet; and
a bridge positioned intermediate the proximal eyelet and the distal eyelet;
wherein:
the flexible element is configured to pass through the proximal eyelet and the distal eyelet to couple the flexible element to the bridge at the third portion of the flexible element engaged with the bridge;
the first and second portions of the flexible element are configured to pass through the anchor along an interior space that is formed between the inner wall of the anchor and a driver channel of a driver engagement member that is placed within the longitudinal anchor passageway, such that the first and second portions of the flexible element exit the proximal end of the anchor; and
the first and second portions of the flexible element are configured to enter into and pass through a longitudinal shaft passageway formed through a driver shaft that is coupled to or integrated with the driver engagement member, such that the first and second portions of the flexible element exit the proximal end of the driver shaft.

17. The assembly of claim 16, wherein the anchor comprises an anchor thread disposed about the anchor body.

18. The assembly of claim 17, wherein at least one of the anchor body and the anchor thread comprises a tapered shape.

19. The assembly of claim 16, wherein the bridge comprises:
a bridge recess formed on an outer surface of the anchor body; and
a bridge protrusion formed on the inner wall within the anchor that transversely protrudes into the longitudinal anchor passageway.

20. The assembly of claim 16, wherein the anchor body comprises at least one fenestration formed therethrough.
